(19) United States
(12) Patent Application Publication
(10) Pub. No.: US 2007/0066904 A1
(43) Pub. Date: Mar. 22, 2007

(54) Device and method for a noninvasive cardiac monitor

(76) Inventors: William P. Wiesmann, Washington, DC (US); Loland Alex Pranger, Gaithersburg, MD (US); Ron Riechers, Dayton, OH (US)

Correspondence Address: BARTUNEK & BHATTACHARYYA, LTD., 10420 LITTLE PATUXENT PARKWAY, SUITE 405, COLUMBIA, MD, 21044-3533, US

(21) Appl. No.: 11/520,441

(22) Filed: Sep. 13, 2006

Related U.S. Application Data
(60) Provisional application No. 60/716,662, filed on Sep. 13, 2005.

(51) Int. Cl. A61B 5/02; A61B 5/04

(52) U.S. Cl. 600/508; 600/509; 600/526

(57) ABSTRACT

The present invention focuses on a method and device for noninvasively measuring cardiac output at a distance, without direct contact to the patient using stepped frequency electromagnetic interrogation. The method detects cardiac versus non-cardiac activity by quantifying the changes in the dielectric properties of blood as it goes through the heart.

### PRIORITY DOCUMENTS

This application claims priority from U.S. Provisional Application 60/716,662, filed Sep. 13, 2005.

### STATEMENT OF GOVERNMENT INTEREST

The invention described herein may be licensed by or for the United States Government, wherein the United States Government may have a nonexclusive, nontransferable, irrevocable, paid-up license to practice or have practiced for or on behalf of the United States the subject invention throughout the world.

### BACKGROUND

Cardiac output, stroke volume multiplied by heart rate, is the benchmark measurement for cardiovascular function. This measurement is traditionally attained through methods such as invasive pressure monitoring via a catheter or noninvasively through an electrocardiogram.

New research shows that noninvasive remote detection has new utility in the medical sciences. Please see Boric-Lubecke et al, Doppler Radar Sensing of Multiple Subjects in Single and Multiple Antenna Systems; Science & Technology Review, Micropower Impulse Radar, January/February 1996; Staderini, An UWB Radar Based Stealthy ‘Lie Detector’; Samardzija, et al., Applications of MIMO Techniques to Sensing of Cardiopulmonary Activity; Folke, et al., Critical Review Of Non-Invasive Respiratory Monitoring In Medical Care, Med. Biol. Eng. Comput., 2003 41, 377-383. The information provided by these publications is incorporated by reference in the present invention.

These new non-invasive procedures have led the inventors of the present invention to provide a non-invasive cardiac monitor as described below.

### SUMMARY

It is an objective of the present invention to provide a non-invasive cardiac monitor that uses pulsed step frequency electromagnetic interrogation to detect changes from the patient's beating heart.

It is yet another objective of the present invention to provide a non-invasive cardiac monitor where the pulsed step electromagnetic interrogation operates in the microwave frequencies.

It is yet another objective of the present invention to provide a non-invasive cardiac monitor that is hand held, and assists a user to quickly triage live/dead status.

It is yet another objective of the present invention to provide a non-invasive cardiac monitor that can be integrated into sports equipment for monitoring persons using the equipment without contact and not requiring disposable or cleaning of any contact leads.

It is yet another objective of the present invention to provide a non-invasive cardiac monitor to allow for cardiac monitoring of multiple patients within a remote location or building.

It is yet another objective of the present invention to provide a non-invasive cardiac monitor that is capable of remotely establishing the position of an individual.

It is yet another objective of the present invention to provide a non-invasive cardiac monitor that utilizes advance signal processing techniques and sufficient computing power, real-time, beat-to-beat analysis of intensity, variability, rate, cardiac output, and other physiologic parameters.

These and other features are described below.

### DESCRIPTION OF THE FIGURES

FIG. 1 is a schematic of the present invention.

### DETAILED DESCRIPTION OF THE EMBODIMENTS

The present invention focuses on a method and device for noninvasively measuring cardiac output at a distance, without direct contact to the patient using stepped frequency electromagnetic interrogation. The method detects cardiac versus non-cardiac activity by quantifying the changes in the dielectric properties of blood as it goes through the heart.

Based upon research conducted by the inventors, the present invention is based upon the capability to measure time varying dielectric properties such as the permittivity of free blood. This was accomplished using a standard vector network analyzer using a stepped frequency excitation of the area of interest and where previously developed signal processing software provides usable output. Due to the availability of miniaturized components and new small, lightweight RF commercial off the shelf (COTS) systems and subsystems, cardiac monitor is now feasible.

The present invention can be used to monitor cardiovascular resuscitation after traumatic injury, during and after surgery, and in critical care units. Because the device is designed to continuously monitor cardiovascular output, this device is capable of use by firefighters and first responders to monitor cardiovascular collapse due to heat stroke and fluid loss.

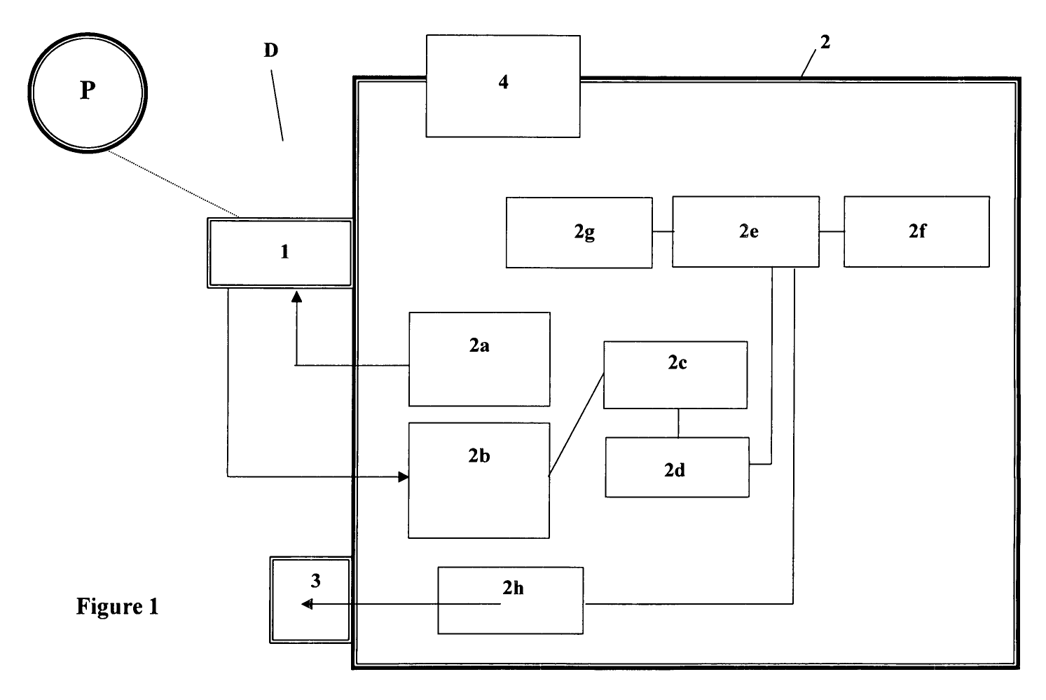

As shown in FIG. 1, the device D includes an antenna 1 that is utilized to pick up medical information on a patient P without direct contact with patient P. In a preferred embodiment, the antenna 1 detects cardiac signals using electromagnetic frequencies, particularly microwave frequencies. The antenna 1 is connected to an interrogator unit 2 that utilizes pulsed step frequency electromagnetic signals to detect dielectric changes from patient P's beating heart. The present invention is based on the fact that different portions of a human heart generate different dielectric signals. For example, the arterial output of the heart generates a different dielectric signature than the surrounding heart area. The dielectric signal reflects the volume of blood leaving the heart , thereby correlating a change in the dielectric signals with the volume of blood flow. A stepped CW frequency generator 2a transmits a step frequency signal to the antenna 1 to collect dielectric signal from the heart at periodic intervals t for the period of t₁ to tₓ. The receiver 2b then receives the signal at periodic intervals r for the period of r₁ to rₓ. The generator 2a transmits a signal t and the receiver 2b receives r for each interval from 1 to x. Each received signal r is send to a signal acquisition 2c and thereafter to signal converter 2d. The signal converter 2d filters, amplifies, processes and converts analog into digital signals for each signal r for the period 1 through x. Thereafter each signal r is sent to processor 2e to generate a data pool of signal information corresponding to the cardiac signature of the patient P. The data may be displayed on display 2f, as individual points corresponding to each signal r, or as a data signature based upon a summation of r for the interval period from 1 through x. Additionally, the data may be stored in memory 2g and/or transmitted to a remote location via data telemetry 2h and antenna 3. The power source 4 is used to operate all necessary components within the interrogator unit 2. The power source may be an internal power source such as batteries or may be an outside power source.

In a preferred embodiment, the device D is a portable, hand held interrogator unit having an attached antenna that is capable of obtaining vital statistics on the patient P.

In another preferred embodiment, the device D can be integrated into sports equipment for monitoring persons without contact and not requiring disposable or cleaning of any contact leads.

It is also understood by one of ordinary skill in the art that the device D of the present invention can change the mode and type of detection in a variety of environments from aerial vehicles to land vehicles to remote geographic locations, by varying the quality of the antenna, by altering signal processing and filtering capabilities and also by changing processor capabilities.

Additionally it is within the scope of the present invention to utilize device D for obtaining information on multiple persons within a building, block, as well as the application of advance signal processing techniques, real-time, beat-to-beat analysis of intensity, variability, rate, cardiac output, and other physiologic parameters.

What is claimed is:

1. A device for noninvasively measuring cardiac output without direct contact to a patient: comprising:
an antenna constructed so as to detect cardiac signals from said patient's beating heart as electromagnetic frequencies;
an interrogator connected to said antenna, wherein said interrogator is constructed so as to utilize pulsed step electromagnetic frequencies and further constructed to detect dielectric changes from said patient's beating heart;
a frequency generator connected to said antenna so as to transmit a stepped frequency signal to said antenna and further constructed so as to collect dielectric signals from said heart at a plurality of first periodic intervals;
a receiver constructed so as to receive said signals from said frequency generator at a plurality of second periodic intervals;
a signal acquisition means constructed so as to receive said plurality signals at said second periodic intervals from said receiver;
a signal converter constructed so as to filter, amplify, process and convert said signals from said signal acquisition means to a plurality of digital signals;
a processor constructed so as to receive each of said plurality of digital signals and further constructed so as to generate a data pool of signal information corresponding to a cardiac signature of said patient;
a display means constructed so as to display said data; and
a power source constructed so as to connect to said antenna, said interrogator, said stepped frequency generator, said receiver, said signal acquisition means, said signal converter, said processor and said display means and operate said device.

2. The device as recited in claim 1 wherein said power source are batteries.

3. The device as recited in claim 2 wherein said device is portable and hand-held, and said antenna further constructed so as to receive vital statistics on said patient.

4. The device as recited in claim 3 wherein said device is constructed so as to be integrated into sports equipment so as to monitor said patient without contact leads.

5. A method for noninvasively measuring cardiac output of a patient at a distance comprising:
(a) detecting cardiac signals from said patient using electromagnetic frequencies using an antenna;
(b) utilizing pulsed step frequency electromagnetic signals and detecting dielectric changes from said patient's beating heart;
(c) transmitting a step frequency signal to said antenna and collecting dielectric signals from said patient's heart at a plurality of first periodic intervals;
(d) receiving signals from said antenna for a plurality of second periodic intervals;
(e) transmitting said received signals to a signal acquisition means and further transmitting said signals to a signal converter;
(f) filtering, amplifying, processing and converting said signals into digital signals;
(g) processing said digital signals and generating a data pool of signal information corresponding to said patient's cardiac signature.

6. A method as recited in claim 5 and further comprising displaying said data pool.

7. A method as recited in claim 5 and further comprising transmitting said data pool to a remote location.

8. A method as recited in claim 6 and further comprising displaying said data pool as individual points corresponding to each said digital signal.

9. A method as recited in claim 6 and further comprising displaying said data pool as a data signature based upon a summation of second periodic intervals.

10. A method as recited in claim 7 and further comprising transmitting said data pool as individual points corresponding to each said digital signal.

11. A method as recited in claim 7 and further comprising transmitting said data pool as a data signature based upon a summation of second periodic intervals.
